(19) United States
(12) Patent Application Publication
(10) Pub. No.: US 2022/0168075 A1
(43) Pub. Date: Jun. 2, 2022

(54) Methods for Improving Intraoral Dental Scans

(71) Applicant: Marc Lemchen, New York, NY (US)

(72) Inventor: Marc Lemchen, New York, NY (US)

(21) Appl. No.: 17/534,873

(22) Filed: Nov. 24, 2021

Related U.S. Application Data
(60) Provisional application No. 63/119,818, filed on Dec. 1, 2020.

(51) Int. Cl. A61C 9/00; G06T 7/00; G06Q 50/04; A61C 13/00

(52) U.S. Cl. CPC A61C 9/0053; G06T 7/0014; G06T 2207/30036; A61C 13/0004; G06Q 50/04

(57) ABSTRACT

A method for improving 3D intraoral scans performed at home which allows the dental professional to directly fill in any missing 3D image data with pre-existing 3D image data that was previously obtained from the patient. After the at home intraoral scan is completed, the patient transmits it to the dental professional who then reviews it and determines whether or not the at home intraoral scan comprises any missing 3D image data required for producing a complete and accurate 3D model. If the self-intraoral scan is complete, the dental professional may transmit it directly to an appliance manufacturer. However, if the at home intraoral scan is incomplete and comprises missing 3D image information, the dental professional may instead complete the provided scan by comparing it with the reference intraoral scan. The improved scan is then sent to the appliance manufacturer who may then produce the required appliance.

### RELATED APPLICATIONS

The present application is a non-provisional of U.S. provisional application Ser. No. 63/119,818, filed on Dec. 1, 2020, which is incorporated herein by reference in its entirety and to which priority is claimed pursuant to 35 USC 119.

### BACKGROUND

### Field of the Technology

The invention relates to methods and apparatus for improving intraoral dental scans, specifically a method which allows a patient to produce high quality and complete intraoral dental scans from home which may be used to fabricate aligners, retainers, or other dental or orthodontic appliances.

### Description of the Prior Art

Currently, when dental appliances such as retainers or a series of aligners are required for a patient, an accurate representation of the patient's teeth is required. Most often this representation is obtained either through a 3D image scan or by taking a physical impression of the patient's teeth.

With regard to 3D image scans, most scans of teeth are done using specialized equipment and software such as those produced by Itero Element® or 3Shape TRIOS® which provide STL data to make aligners, retainers, and other dental appliances. While these are amazingly accurate devices, they require trained individuals to operate them when scanning a patient's teeth. Additionally, because these type of 3D scans require relatively large pieces of equipment and trained operators, the patient must physically travel to an office each time a scan is required which is not only inconvenient but which can further expose the patient and the operator to COVID-19 and other transmittable illnesses. However, the scans that are produced far exceed the accuracy of physical dental impressions, including those taken by patients at home, often for do-it-yourself aligners or retainers through services such as Smile Direct Club®.

Alternatively, many patients are trying to use smart device technology to scan their teeth at home in place of a self-impression done with a kit which is mailed to them. For example, after downloading an application to their smart device, the patient angles the built-in camera towards their teeth and begins to pan the smart device around their mouth as dictated by a set of instructions relayed to the patient via the app. In some instances, the patient is provided with a mouth prop or bite block to keep their mouth open while the scan is performed. The smart device itself is placed into a custom frame with enlarged handles so that the patient can more easily hold and complete the scanning process. Other attachments or scanning devices may be used in conjunction with or which couple to the smart device, either directly via a plug or cord or wirelessly via a Bluetooth® or internet connection. The issue with many of these at home scans however is how well the scan works and how accurate the final scan is. Even the Itero Element® scans require the operator to go back and fill in voids and missed areas. As of yet, artificial intelligence has not yet been used to fill in these missing spots accurately.

What is needed is a method which allows a patient to obtain an accurate 3D scan of their teeth without having to physically travel to a dentist's office each time a new scan or dental appliance is required.

### BRIEF SUMMARY

The current invention provides a method for improving intraoral scans. The method includes performing an intraoral scan to produce a reference image, performing an at home or self-intraoral scan to produce a secondary image, and then determining if the secondary image includes any missing image data. Next, the secondary image is improved using that image data from the reference image to produce an improved image if it has been determined that the secondary image has any missing image data. Alternatively, the secondary image is transmitted to an appliance manufacturer if it has been determined that the secondary image does not in fact have any missing image data.

In one embodiment, the method also includes transmitting the improved image to the appliance manufacturer.

In another embodiment, improving the secondary image using data from the reference image in order to produce an improved image specifically includes inserting image data that has been removed from the reference image into the secondary image.

In yet another embodiment, the method further includes storing the reference image, the secondary image, and the improved image in a single or common memory storage. Here, the reference image stored in the memory storage may then be replaced or updated with the improved image, preferably by replacing the reference image stored in the memory storage with the secondary image.

In a further embodiment, inserting image data removed from the reference image into the secondary image specifically involves inserting the image data removed from the reference image in at least one location within the secondary image where it has been determined that such image data is missing.

In a different embodiment, the method also includes transmitting the secondary image to a processor or sever that is in the control of an operator such as a dentist, orthodontist, or other dental professional. Additionally, a visual indicator may be automatically added or attached to the secondary image thereby notifying the operator to review the secondary image.

In a further embodiment, the method includes transmitting the secondary image directly to a memory storage.

The current invention also provides a method for a patient in communication with an operator to improve intraoral scans. The method includes performing an intraoral scan by the operator so as to produce a reference image of at least one portion of the patient's mouth, performing a self-intraoral scan by the patient so as to produce a secondary image of the at least one portion of the patient's mouth, and then transmitting the secondary image to a processor or server which is accessible by both the operator and the patient, the processor or server itself comprising a memory storage. Next, it is determined if the received secondary image comprises any missing image data. If the secondary image in fact does have missing image data, the secondary image is then improved using data from the reference image in order to produce an improved image. If however the secondary image does not have any missing image data, the secondary image is instead transmitted to an appliance manufacturer.

In one embodiment, determining whether or not the secondary image has any missing image data specifically includes comparing the secondary image to the reference image by the operator.

In another embodiment, the reference image and the improved image are stored or otherwise sent to the memory storage within the processor or server by the operator. Additionally, the reference image stored to the memory storage may then be replaced by the improved image.

In yet another embodiment, the method also includes transmitting the improved image to the appliance manufacturer by the operator.

In another embodiment, improving the secondary image using data from the reference image in order to produce an improved image specifically includes the operator inserting image data that has been removed from the reference image into at least one portion of the secondary image.

In a further embodiment, the method also includes providing the patient with a set of instructions for performing the self-intraoral scan by the patient in order to produce the secondary image of the at least one portion of the patient's mouth.

In one particular embodiment, transmitting the secondary image to the processor or server which is accessible by both the operator and the patient includes automatically adding or attaching a visual indicator to the secondary image thereby notifying the operator to review the secondary image.

In another embodiment, the method also includes performing an intraoral scan by the operator in order to produce a plurality of reference images of a corresponding plurality of different portions of the patient's mouth and performing a self-intraoral scan by the patient in order to produce a plurality of secondary images of a corresponding plurality of portions of the patient's mouth. Next it is determined if any one of the plurality of secondary images comprises any missing image data. When such missing image data is detected, the plurality of secondary images are then improved by using data from the plurality of references images in order to produce a plurality of improved images. This embodiment may also include replacing at least one of the plurality of reference images with at least one of the plurality of improved images so as to provide an updated record of the patient's treatment progress.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a flow chart demonstrating a main embodiment of the current invention.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

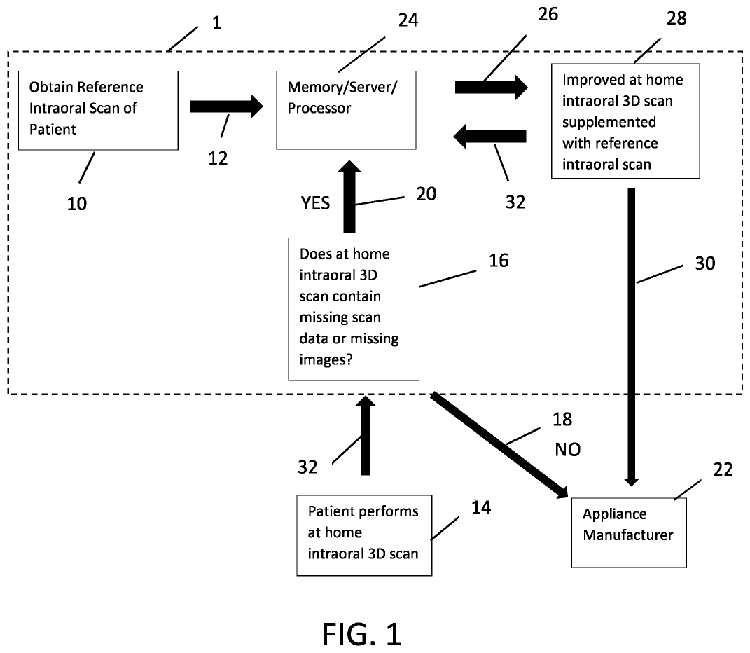

In a first embodiment of the current invention, a method is provided for improving 3D intraoral scans performed at home which allows the dental professional to directly fill in any missing 3D image data with pre-existing 3D image data that was previously obtained from the patient. Referring to FIG. 1, an operator such as a dentist, orthodontist, or other dental technician performs an in-office highly accurate 3D intraoral scan of a patient using a 3D intraoral scanning system such as the Itero Element® or 3Shape TRIOS® to produce a reference intraoral scan 10. Once completed, the reference 10 scan is sent in step 12 to a memory, server, processor, or other device/location 24 which is accessible by both the operator and the patient via a wireless network such as the internet.

Next, the patient while at home performs a secondary scan 14 using a camera, smart phone, tablet, or any other smart device which comprises the ability to record an image or a series of images and can connect to an external network such as the internet. The patient performs the intraoral scan according to a set of instructions offered by the operator who may have previously provided the patient with a written set of instructions or who may be video conferencing with the patient so as to give step by step instructions in real time as the patient progresses through the scanning process. Alternatively, an application or tutorial program running on the patient's smart device may provide the patient audio and/or video instructions while the intraoral scan is performed. After the at home or self-intraoral scan 14 is completed, the patient transmits it to the operator in step 32 who then reviews it in step 16 and determines whether or not the self-intraoral scan 14 comprises any missing 3D image data such as missing or incomplete surfaces of teeth, gingiva tissue, or other features required for producing a complete and accurate 3D model. If the self-intraoral scan 14 is complete and contains all the necessary 3D image data, the operator transmits the self-intraoral scan 14 directly to an appliance manufacturer 22 in step 18. However, as is far more likely the case, if the self-intraoral scan 14 is incomplete, comprises missing 3D image information or “holes,” or otherwise comprises any differences between it and the reference scan 12, the operator sends the self-intraoral scan 14 to the same processor/server 24 comprising the reference intraoral scan 10 at step 20.

In an alternative embodiment, the application, program, or computer software stored on the patient's smart device and running the scan may automatically determine if there is any missing STL data in the at home or self-intraoral scan 14 and then automatically send it to the operator with a flag or other visual indicator coupled to the self-intraoral scan 14 for the operator to further review the self-intraoral scan 14. The operator may then confirm whether or not the self-intraoral scan 14 does indeed comprise any missing 3D data and then transmit the self-intraoral scan 14 to either the appliance manufacturer 22 or the server/processor 24 accordingly.

In yet a further embodiment, the application, program, or computer software stored on the patient's smart device and running the scan may automatically send the self-intraoral scan 14 to the processor/server 24 regardless, thus skipping the need for the operator to review or confirm any details about the self-intraoral scan 14 entirely.

Once the processor/server 24 has both the self-intraoral scan 14 and the reference intraoral scan 10, the processor/server 24 uses the less accurate self-intraoral scan 14 to check the progress of the patient's treatment and rescan for new aligners. The processor/server 24 takes the reference intraoral scan 10 taken during the initial office visit and uses it to fill in, interpolate, or otherwise complete the voids or any other missing 3D image or STL data in the self-intraoral scan 14. In one particular embodiment, this would be specifically accomplished by taking the self-intraoral scan 14 and comparing it or referencing it against the reference intraoral scan 10 to fill in the voids by inserting the missing image data removed from the reference intraoral scan 10 into at least one appropriate location within the self-intraoral scan 14. For example, if half the lingual surface of the first molar is missing from the at home intraoral scan 14, the program stored within the processor/server 24 will use the 3D image data it has on that tooth as taken from the reference intraoral scan 10 and applies or inserts the missing 3D image data into the self-intraoral scan 14 at step 26, thus completing the self-intraoral scan 14 of the tooth to produce a final and accurate improved image 28. In other words, the secondary image is used to locate several clearly recognizable points on individual teeth, such as the occlusal anatomy, grooves, defects, restorations, which is then refined using the “exact” image data from the reference scan to perfect the image of each tooth. The location of each tooth, however, is established from the secondary image which shows the location and relationship of each tooth related to any adjacent and opposing teeth and within the dental arch. Because the position of the teeth is changing over the course of an aligner treatment, the shape of the teeth does not change unless the tooth has been damaged or restored since the initial reference scan. The secondary image shows locations and relationships of each tooth to one another, while the reference image provides the exact shape and dimension of each tooth in three dimensions. The secondary images marks each tooth in space, while the reference image provides the shape of the tooth. This process could be repeated for the whole mouth, thereby producing an accurate scan from the limited data originally acquired by the self-intraoral scan 14. The operator in possession of or with access to the processor/server 24 can then send the improved image 28 in step 30 to the appliance manufacturer 22 who can then use the improved image 28 as needed, for example when constructing retainers, dental aligners, or any other dental appliance requiring 3D imaging of the patient.

As further seen in FIG. 1, the operator is in control or possession of, or otherwise maintains access to the processor/server 24, the reference intraoral scan 10, the improved image 28, and the interactions or communication therebetween as represented by the dotted outline 1 surrounding said elements. In other words, the patient sending or transmitting their completed self-intraoral scan 14 and the receipt of either the self-intraoral scan 14 and/or the improved image 28 by the appliance manufacturer 22 represent the input and output, respectively, of the current method which is otherwise under the purview or control of the operator.

In a related embodiment, once an improved image 28 has been completed, the operator may direct the processor/server 24 in step 32 to either supplement or replace the reference intraoral scan 10 with the improved image 28, thereby effectively updating or replacing the reference intraoral scan 10 for future 3D image data correction as the treatment of the patient progresses. For example, if the patient requires a new set of aligners, the patient performs a new or second self-intraoral scan 14 which is then compared and supplemented by the improved image 28 within the processor/server 24, thereby producing a brand new 3D image representing the current state of the patient's mouth. By continually updated or replacing the reference intraoral scan 10, the appliance manufacturer 22 is always provided with the most up to date patient information and thus can always produce the prescribed dental appliance for the patient. It can also be seen that by constantly improving or updating the reference intraoral scan 10, the patient is not required to repeatedly return to the operator's office each time a new scan is needed. Instead, after completing the initial scan the patient may remain at home for the duration of their treatment, no matter how many dental appliances the patient may ultimately need.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

I claim:

1. A method for improving intraoral scans comprising:
performing an intraoral scan to produce a reference image;
performing a self-intraoral scan to produce a secondary image;
determining if the secondary image comprises any missing image data;
improving the secondary image using data from the reference image to produce an improved image if it has been determined that the secondary image comprises missing image data; or
transmitting the secondary image to an appliance manufacturer if it has been determined that the secondary image does not comprise missing image data.

2. The method of claim 1 further comprising transmitting the improved image to the appliance manufacturer.

3. The method of claim 1 where improving the secondary image using data from the reference image to produce an improved image if it has been determined that the secondary image comprises missing image data comprises inserting image data removed from the reference image into the secondary image.

4. The method of claim 1 further comprising storing the reference image, the secondary image, and the improved image in a memory storage.

5. The method of claim 4 further comprising replacing the reference image stored in the memory storage with the improved image.

6. The method of claim 1 where inserting image data removed from the reference image into the secondary image comprises inserting the image data removed from the reference image in at least one location within the secondary image where it has been determined that image data is missing.

7. The method of claim 4 further comprising further comprising replacing the reference image stored in the memory storage with the secondary image.

8. The method of claim 1 further comprising transmitting the secondary image to a processor or sever in the control of an operator.

9. The method of claim 8 where transmitting the secondary image to the processor or sever in the control of an operator comprises automatically coupling a visual indicator to the secondary image notifying the operator to review the secondary image.

10. The method of claim 1 further comprising transmitting the secondary image directly to a memory storage.

11. A method for a patient in communication with an operator to improve intraoral scans, the method comprising:
performing an intraoral scan by the operator to produce a reference image of at least one portion of the patient's mouth;
performing a self-intraoral scan by the patient to produce a secondary image of the at least one portion of the patient's mouth;
transmitting the secondary image to a processor or server accessible by both the operator and the patient, the processor or server comprising a memory storage;
determining if the secondary image comprises any missing image data;
improving the secondary image using data from the reference image to produce an improved image if it has been determined that the secondary image comprises missing image data; or
transmitting the secondary image to an appliance manufacturer if it has been determined that the secondary image does not comprise missing image data.

12. The method of claim 11 where determining if the secondary image comprises any missing image data comprises comparing the secondary image to the reference image by the operator.

13. The method of claim 11 further comprising storing the reference image and the improved image to the memory storage within the processor or server by the operator.

14. The method of claim 11 further comprising transmitting the improved image to the appliance manufacturer by the operator.

15. The method of claim 13 further comprising replacing the reference image stored to the memory storage with the improved image.

16. The method of claim 11 where improving the secondary image using data from the reference image to produce an improved image if it has been determined that the secondary image comprises missing image data comprises the operator inserting image data removed from the reference image into at least one portion of the secondary image.

17. The method of claim 11 further comprising providing the patient with a set of instructions for performing the self-intraoral scan by the patient to produce the secondary image of the at least one portion of the patient's mouth.

18. The method of claim 11 where transmitting the secondary image to the processor or server accessible by both the operator and the patient comprises automatically coupling a visual indicator to the secondary image notifying the operator to review the secondary image.

19. The method of claim 11 where performing an intraoral scan by the operator to produce a reference image of at least one portion of the patient's mouth comprises performing an intraoral scan by the operator to produce a plurality of reference images of a corresponding plurality of different portions of the patient's mouth,
where performing a self-intraoral scan by the patient to produce a secondary image of the at least one portion of the patient's mouth comprises performing a self-intraoral scan by the patient to produce a plurality of secondary images of a corresponding plurality of portions of the patient's mouth,
where determining if the secondary image comprises any missing image data comprises determining if any one of the plurality of secondary images comprises any missing image data, and
where improving the secondary image using data from the reference image to produce an improved image if it has been determined that the secondary image comprises missing image data comprises improving the plurality of secondary images using data from the plurality of references images to produce a plurality of improved images.

20. The method of claim 19 further comprising replacing at least one of the plurality of reference images with at least one of the plurality of improved images.
